(19) United States
(12) Patent Application Publication
(10) Pub. No.: US 2009/0221637 A1
(43) Pub. Date: Sep. 3, 2009

(54) SOLID-STATE SALT ARGATROBAN FORMULATIONS AND METHODS FOR PRODUCING AND USING THE SAME

(75) Inventor: George Owoo, North Plainfield, NJ (US)

Correspondence Address: K&L Gates LLP, P.O. Box 1135, Chicago, IL, 60690-1135, US

(73) Assignees: BAXTER INTERNATIONAL INC., Deerfield, IL (US); BAXTER HEALTHCARE S.A., Zurich (CH)

(21) Appl. No.: 12/040,768

(22) Filed: Feb. 29, 2008

(51) Int. Cl. A61K 31/4709; A61L 33/04; A61P 7/02

(52) U.S. Cl. 514/314; 427/2.24

(57) ABSTRACT

Argatroban formulations and methods of making and using the formulations are provided. In an embodiment, the present disclosure provides a solid-state salt formulation of argatroban. Upon reconstitution, the solid-state argatroban forms a solution that can be essentially free of particles and suitable for administration. The solid-state argatroban can be in a crystalline and/or amorphous form. The solid-state argatroban can be packaged in a sealed container that may be aseptically-filled to reduce the microbiological burden of the formulation. The solid-state argatroban can be reconstituted with a solution and administered to persons needing same.

### BACKGROUND

The present disclosure relates to new solid-state formulations of 1-[5-[(aminoiminomethyl)amino]-1-oxo-2-[[(1,2,3,4-tetrahydro-3-methyl-8-quinolinyl) sulfonyl]amino]pentyl]-4-methyl-2-piperidinecarboxylic acid hydrate, commonly known by the generic name “argatroban.” Argatroban is a synthetic direct thrombin inhibitor derived from L-arginine and is a useful anti-coagulant and anti-thrombotic agent.

Argatroban is considered slightly to very slightly soluble in water according to the USP classification of solutes, with solubility on the order of 0.8 to 0.9 mg/mL. It is also both light and heat-sensitive and tends to degrade unless stabilized. Argatroban is commercially available in concentrated form in an aseptically-filled vial containing, per mL, 100 mg argatroban, 750 mg D-sorbitol and 1000 mg dehydrated alcohol.

### SUMMARY

Argatroban formulations and methods of making and using the formulations are provided. In a general embodiment, the present disclosure provides a solid-state salt formulation of argatroban (hereinafter “solid-state argatroban”) suitable for human and pharmaceutical use. The solid-state argatroban can be made by lyophilization (freeze drying) of an argatroban “ionic liquid” and/or from in-situ salts of argatroban formed by crystallization, precipitation, and lyophilization techniques. The solid-state argatroban maintains its isomeric content and can be transformed into oral dosage forms, injections, topical treatments, etc., by mixing with the appropriate excipients. For injections, the solid-state argatroban can be in a powder form that can be redissolved into solution diluents, which can be essentially free of visible particles and free of dehydrated alcohol. The reconstituted argatroban solution made from the solid-state argatroban may optionally further contain a buffering agent to help maintain pH and an osmotic-adjusting agent to enhance infusion properties. The solid-state argatroban can have an extended shelf life, for example, greater than about 12 months.

The argatroban solutions made from the solid-state argatroban can also be storage-stable (both light and heat), capable of being aseptically-filled and/or sterilized (e.g. heat), and contain the argatroban in a range of concentrations.

In an embodiment, the solid-state argatroban can be placed in a vial (e.g. 10 mL vial) in an amount ranging between about 1 mg/vial to about 500 mg/vial.

In an embodiment, the solid-state argatroban comprises a crystalline form.

In an embodiment, the solid-state argatroban comprises an amorphous form.

In an embodiment, the solid-state argatroban comprises a mixture of crystalline and amorphous form.

In an embodiment, the solid-state argatroban comprises a stable powdered form of argatroban with the isomeric composition range from 60:40±5% R:S

In an embodiment, the argatroban “ionic liquid” comprises a pH ranging from about 3.5 to about 6.5.

In an embodiment, the argatroban “ionic liquid” comprises a pH ranging from about 4.5 to about 6.5.

In an embodiment, the argatroban “ionic liquid” comprises a bulking agent selected from the group consisting of lactose, manitol maltose, sucrose, dextrans, glycine, sorbitol, glucose, calcium, non-reducing sugars and combinations thereof.

In an embodiment, the reconstituted argatroban solution comprises an osmotic agent selected from the group consisting of sodium chloride, calcium chloride, potassium chloride, dextrose, sodium lactate and combinations thereof.

In an embodiment, the argatroban “ionic liquid” and/or the reconstituted argatroban solutions are sterilized.

In another embodiment, the present disclosure provides a reconstituted argatroban solution comprising a solid-state argatroban dissolved in an aqueous solution. The solid-state argatroban comprises an X-ray diffraction pattern having a characteristic peak expressed in d-values (Å) selected from the group consisting of: 19.46, 9.74, 8.63, 7.42, 6.99, 6.50, 6.02, 5.53, 5.35, 5.20, 4.86, 4.71, 4.45, 4.17, 3.99, 3.88, 3.56, 3.44, 3.36, 3.22, 2.88.

In yet another embodiment, the present disclosure provides a compound comprising a solid-state argatroban. For example, the argatroban composition can comprise an X-ray diffraction pattern having a characteristic peak expressed in d-values (Å) selected from the group consisting of: 19.46, 9.74, 8.63, 7.42, 6.99, 6.50, 6.02, 5.53, 5.35, 5.20, 4.86, 4.71, 4.45, 4.17, 3.99, 3.88, 3.56, 3.44, 3.36, 3.22, 2.88.

In an alternative embodiment, the present disclosure provides a method for producing an argatroban solution. The method comprises dissolving a solid-state argatroban in an aqueous solution to form the solution. The solution has an argatroban concentration ranging between about 1 μg/mL to about 500 mg/mL.

In another embodiment, the present disclosure provides a method for producing a medical product. The method comprises providing a solution comprising a solid-state argatroban dissolved in an aqueous solution and applying the solution to a surface of a medical device.

In an embodiment, the aqueous solution comprises an argatroban concentration ranging between about 1 μg/mL to about 500 mg/mL

In an embodiment, the medical product is selected from the group consisting of a tablet, a transdermal patch, a stent, a polymeric substrate, and combinations thereof.

In an embodiment, the method comprises drying the solution and forming a coating of argatroban on the medical device.

In yet another embodiment, the present disclosure provides a method of administering argatroban to a person. The method comprises preparing a solution comprising a solid-state argatroban dissolved in an aqueous solution, diluting the solution and administering the solution to the person. The solution can have an argatroban concentration ranging between about 1 μg/mL to about 500 mg/mL.

In an embodiment, the solution is sterilized before administering to the person.

In still another embodiment, the present disclosure provides a method of providing anticoagulation and/or anti-thrombotic therapy to a person. The method comprises administering to a person in need of same a solution comprising a solid-state argatroban dissolved in an aqueous solution. The solution can have an argatroban concentration between about 1 μg/mL to about 500 mg/mL.

In an embodiment, the method comprises adding a bulking agent to the dissolved argatroban solution before filtering the dissolved argatroban solution.

In an embodiment, the bulking agent is selected from the group consisting of sucrose, lactose, manitol and combinations thereof.

In an embodiment, the method comprises dissolving the solid-state argatroban in an aqueous solution to form an argatroban solution.

An advantage of the present disclosure is to provide improved formulations comprising argatroban.

Another advantage of the present disclosure is to provide sterilized solutions comprising argatroban.

Yet another advantage of the present disclosure is to provide stable solutions comprising argatroban.

Still another advantage of the present disclosure is to provide improved methods of making formulations comprising argatroban.

Another advantage of the present disclosure is to provide ready to use solutions comprising a sterilized and stable amount of solid state argatroban.

Additional features and advantages are described herein, and will be apparent from, the following Detailed Description and the figures.

### BRIEF DESCRIPTION OF THE FIGURES

FIG. 1 illustrates an X-ray diffraction pattern for a crystalline form of argatroban in free base form.

FIG. 2 illustrates an X-ray diffraction pattern for another crystalline form of argatroban in free base form.

FIG. 3 illustrates an X-ray diffraction pattern for a crystalline hydrochloride salt form of argatroban.

FIG. 4 illustrates an X-ray diffraction pattern for an amorphous acetate salt form of argatroban.

### DETAILED DESCRIPTION

The present disclosure relates to formulations comprising argatroban and methods of making and using the argatroban formulations. In a general embodiment, the present disclosure provides a composition comprising a solid-state argatroban. Upon being reconstituted in suitable solution (e.g. aqueous solution, injection diluent, etc.), the resultant argatroban solution can be free of particles and be suitable for human administration (e.g. intravenous administration). In an embodiment, the solid-state argatroban can be packaged in a sealed container that may be aseptically-filled to reduce the microbiological burden of the formulation. The solid-state argatroban can be stable against oxidative degradation and other adverse chemical reactions, and when packaged appropriately, for example, with amber containers, non-light transmitting polymer containers or aluminum overpouches, can be stable against photolytic degradation.

Free base argatroban is an insoluble compound in water and other neat solvents. As a result, pharmaceutical compositions necessitating free base argatroban at high concentrations require solubility enhancers and/or stabilizers to produce a suitable delivery system for the efficacious and safe argatroban product.

In alternative embodiments, the present disclosure is directed to lyophilized argatroban “ionic liquid” and/or in-situ salts of argatroban made by precipitation and lyophilization techniques. For example, an argatroban drug solid (i.e. free base form) can be converted into a solid-state salt via an in-situ salt formation using dilute inorganic and organic acids. The reaction of salting-in is facilitated mainly by the N-terminus of the L-arginine moiety that is practically slightly soluble at natural pH. The salt solutions can be stable at lower pH. Employing lyophilization techniques can provide solid-state salt formulations having adequate stability. Additionally, by using a suitable combination of co-solvents with the in-situ salt solution, precipitation of the solid-state argatroban can occur. As a result, pharmaceutical compositions comprising the solid-state salt argatroban formulations may posses improved dissolution profiles (reconstitution) due to the solubility of the in-situ salt.

In an embodiment, the reconstituted argatroban solutions comprising the dissolved solid-state argatroban include one or more added dilute acids to the solution in order for the solid-state argatroban to be sufficiently dissolved within the solution. In another embodiment, the argatroban solutions comprising the dissolved solid-state argatroban includes one or more added solvents to the solution in order for the solid-state argatroban to be sufficiently dissolved within the solution. Nevertheless, the argatroban solutions can comprise the same concentration as corresponding free base argatroban and the isomeric composition remains unchanged in the solutions that use acid or solvents/co-solvents to increase the free base argatroban solubility in the solution.

The solid-state argatroban can have different crystallizing natures arising from the precipitated forms of anhydrous and hydrate forms of the argatroban free base. These solid-state salt argatroban formulations can be characterized, for example, by analysis methods such as X-ray diffraction (XRD), CuKα radiation, Thermogravimetric analysis (TG), Karl-Fischer titration and Differential scanning calorimetry (DSC). In an embodiment, the solid-state argatroban comprises an X-ray diffraction pattern having a characteristic peak expressed in d-values (Å) selected from the group consisting of: 19.46, 9.74, 8.63, 7.42, 6.99, 6.50, 6.02, 5.53, 5.35, 5.20, 4.86, 4.71, 4.45, 4.17, 3.99, 3.88, 3.56, 3.44, 3.36, 3.22, 2.88.

In an embodiment, the solid-state argatroban comprises a crystalline form. In another embodiment, the solid-state argatroban comprises an amorphous form. The solid-state salt formulations of argatroban can also be a powdered, granular or other suitable form.

The solid-state salt argatroban formulations can be dissolved in any suitable solution such as water for use during parenteral administration. For example, the argatroban solution can comprise the dissolved solid-state argatroban in a concentration ranging between about 0.5 μg/mL to about 500 mg/mL. In another embodiment, argatroban solution can comprise the dissolved solid-state argatroban in a concentration ranging between about 1 mg/mL to about 250 mg/mL.

The solid-state argatroban of the present disclosure can be packaged in a sealed container and be rendered sterile by aseptic processes. The solid-state argatroban can also be packaged in any suitable containers known in the art such as, for example, vials, syringes, bags, bottles and ampoules. The containers can be fabricated from glass or from any other suitable polymeric materials. The reconstituted solid-state argatroban from the powder can be ready-to-use formulations are typically packaged in vials, syringes, infusion bags and bottles.

In an alternative embodiment, the present disclosure provides a method of making a solid-state argatroban. The method comprises dissolving an acid in an aqueous solution, dissolving argatroban in free base form in the acidified aqueous solution, filtering the dissolved argatroban solution and freeze drying the filtered argatroban solution to form the solid-state argatroban. The method can also comprise adjusting the pH of the dissolved argatroban solution to between 4.0 and 5.0 before filtering the dissolved argatroban solution. The final produced solid-state argatroban can be subsequently dissolved in any suitable solution such as an aqueous solution to prepare an administrable argatroban solution.

A bulking agent can be added to the dissolved argatroban solution before filtering the dissolved argatroban solution. The bulking agent can be, for example, lactose, sucrose, manitol, sugars, non-reducing sugars or combinations thereof.

The argatroban solutions prior to which the solid-state argatroban has been formed (e.g. argatroban “ionic liquid”) can comprise any suitable pH. For example, the argatroban solutions can comprise a pH ranging from about 3.5 to about 8.5. In another embodiment, the argatroban “ionic liquid” comprises a pH ranging from about 4.5 to about 6.5.

The argatroban solutions prior to which the solid-state argatroban has been formed can comprise other suitable components. For example, the argatroban solutions can comprise one or more osmotic buffering agents and/or osmotic and bulking agents. Suitable physiologically-acceptable buffering agents include acetate, glutamate, citrate, tartrate, benzoate, lactate, malate, gluconate, phosphate and glycine, with acetate being preferred. A preferred buffering system comprises a combination of sodium acetate and acetic acid. Buffering agents are present in the solution in a concentration that depends from the concentration of argatroban. The concentration will typically range from 0.05 to 200 mM and from 10 to 100 mM for formulations containing 0.5 μg/mL to 500 mg/mL argatroban.

Suitable osmotic-adjusting agents, when used, can be compatible with the pH requirements of the present formulation, and include one or more of sodium chloride, calcium chloride, potassium chloride, dextrose and sodium lactate. Preferred osmotic-adjusting agents are sodium chloride and dextrose. In alternative embodiments, the argatroban solutions may contain from 1 to 100 mg/mL osmotic-adjusting agent; preferably 4 to 60 mg/mL sodium chloride, more preferably 4 to 10 mg/mL sodium chloride; or dextrose in an amount up to 5% (weight by weight), typically in an amount ranging from 25 to 60 mg/mL. In a further embodiment, the buffering agent and the osmotic agent may be the same composition.

Transforming the acidic argatroban solutions into solid-state salt formulations can achieved using any suitable lyophilization techniques, for example, which can include annealing steps to promote crystallizing formation of hydrate. Development of lyophilization cycles can be aided by DSC to understand the glass transition temperature (Tg) of the freeze-concentrated solutions. The obtained Tg can range between −35° to −20° C. The degree of crystallinity and the lack thereof of powdered solution can be influenced by the presence of salts, the excipients and lyophilization cycles to achieve crystalline material. The resulting argatroban solids of an amorphous nature, crystalline nature and/or a mixture thereof can be diluted and used for parenteral administration.

The stability of the reconstituted solid-state salt argatroban formulations may be dependent on the pH of the solution and exposure to light. In true aqueous solutions of argatroban, maximum stability can be found in a broader pH range from 3.5 to 8.5 with optimal conditions centered between pH 4.5 and 6.5. The stability of the argatroban solid/powder can also be dependent on the type of salt solutions the solid/powder was created from, the specific lyophilization process used and other characteristics of the resultant materials.

In the present specification, the term “stable” means remaining in a state or condition that is suitable for administration to a patient. Formulations according to the present disclosure are found to be stable when maintained at room temperature for at least 24 months, and are generally stable at room temperature for 24 to 36 months.

in the present specification, the term “sterile” composition or solution means a composition or solution that has been brought to a state of sterility and has not been subsequently exposed to microbiological contamination, (e.g. the container holding the sterile composition or solutions has not been compromised). Sterile compositions or solutions are generally prepared by pharmaceutical manufacturers in accordance with current Good Manufacturing Practice (“cGMP”) regulations of the U.S. Food and Drug Administration.

The solid-state argatroban in embodiments of the present disclosure can take the form of a sterile, stable, argatroban powder and upon reconstitution result in a ready-to-use formulation for infusion. Specific devices such as the Advantagemate™, SOLOMIX®, etc., can be used for reconstitution. This avoids the inconvenience of diluting a concentrated argatroban small volume parenteral formulation into infusion diluents prior to infusion, as well as eliminates the risk of microbiological contamination during aseptic handling and any potential calculation or dilution error. Nevertheless, the solid-state argatroban can also be used to form a concentrated argatroban formulation that can be diluted prior to administration.

The reconstituted sterile, stable argatroban solutions in embodiments of the present disclosure can be suitable for parenteral administration to a patient. For example, the solution may be administered in the form of a bolus injection or intravenous infusion. Suitable routes for parenteral administration include intravenous, subcutaneous, intradermal, intramuscular, intraarticular, and intrathecal. The ready-to-use formulation of the disclosure is preferably administered by intravenous infusion. The argatroban solution can be diluted and then administered to the person. The administered argatroban solution can have an argatroban concentration ranging between about 0.5 μg/mL to about 500 mg/mL. The solution can also be sterilized before administering to the person.

In still another embodiment, the present disclosure provides a method of providing anticoagulation and/or antithrombotic therapy to a person. The method comprises administering to a person in need of same a solution comprising a solid-state argatroban dissolved in an aqueous solution. The administered solution can have an argatroban concentration between about 0.5 μg/mL to about 500 mg/mL.

The solid-state argatroban according to an embodiment of the present disclosure can be prepared into small volume parenteral (SVP) and admixed to large volume parenteral (LVP) dosage forms. The dosage forms can be held in any suitable container. Suitable containers include, for example, glass or polymeric vials, ampoules, syringes or infusion bags with sizes ranging from 1 ml to 500 ml. SVP ready-to-use solutions are typically filled into ampules and vials in 1 to 100 mL presentations. In addition, syringes can be used as the container for a ready-to-use SVP, which are sold as “pre-filled syringes”. The LVP presentations can be contained in infusion bags or bottles.

Polymeric containers are preferably flexible and can contain or be free of polyvinylchloride (PVC). Preferred containers are free of PVC, such as those disclosed in U.S. Pat. Nos. 5,849,843 and 5,998,019, the entire disclosures of which are herein incorporated by reference.

Sterile reconstituted argatroban solutions according to the present disclosure may be prepared using aseptic processing techniques. Aseptic filling is ordinarily used to prepare drug products that will not withstand heat sterilization, but in which all of the ingredients are sterile. Sterility is maintained by using sterile materials and a controlled working environment. All containers and apparatus are sterilized, preferably by heat sterilization, prior to filling. The container (e.g., vial, ampoule, infusion bag, bottle, or syringe) are then filled under aseptic conditions.

In alternative embodiments, the reconstituted argatroban solutions made from the solid-state argatroban can comprise one or more acids, for example, to provide pH adjustment. Nonlimiting examples of suitable acids for dissolving the argatroban include phosphoric acid, acetic acid, tartaric acid, citric acid, formic acid, malic acid, hydrochloric acid and combinations thereof. The acids can be typically employed in the solution at concentrations ranging from 0.01 to 3 N, depending on the degree of ionization and association of the counter-ion stability in an aqueous environment. In an embodiment, the acetic acid is glacial acetic acid having a normality from about 2N to about 10N. In an alternative embodiment, the pH of the solution may be from about 1.0 to about 3.5 or any value therebetween.

In an alternative embodiment, the reconstituted argatroban solution includes one or more solvents. Nonlimiting examples of suitable solvents include ethyl acetate, methylene chloride, propylene glycol, a polyethylene glycol having a molecular weight from about 100 to about 1000 or any molecular weight therebetween, benzyl alcohol and combinations thereof. In an embodiment, the polyethylene glycol has a molecular weight from about 200 to about 400.

In another embodiment, the reconstituted argatroban solution utilizes a co-solvent (i.e., at least two solvents) matrix. For example, the solution includes argatroban dissolved in a first solvent that is ethyl acetate, methylene chloride, propylene glycol or polyethylene glycol and a second solvent. The solution can have an argatroban concentration from about 1 mg/mL to about 500 mg/mL. The second solvent may be a propylene glycol or polyethylene glycol that is different than the first or benzyl alcohol. In a further embodiment, the first solvent may be a polyethylene glycol having a first molecular weight and the second solvent may be a polyethylene glycol having a molecular weight different than the molecular weight of the first polyethylene glycol. The solution may also include an acid as previously disclosed herein.

In yet another embodiment, an acid, a base, or a buffering agent may be added to the reconstituted solution to maintain the pH from about pH 2.3 to about pH 8.5 or from about pH 4.5 to about pH 6.5. Nonlimiting examples of suitable buffering agents include acetate, glutamate, citrate, tartrate, benzoate, lactate, malate, gluconate, phosphate, glycine and combinations thereof. In an embodiment, the buffering agent may be a salt of the acid. For example, the acid may be acetic acid and the buffering agent may be sodium acetate. Other nonlimiting acid-buffering agent combinations include tartaric acid-sodium tartrate, citric acid-sodium citrate, formic acid-sodium formate, malic acid-sodium malate, hydrochloric acid-sodium chloride, benzoic acid-sodium benzoate, glutamic acid-sodium glutamate, lactic acid-sodium lactate, gluconic acid-sodium gluconate, phosphoric acid-sodium phosphate and combinations thereof. It is understood that any of the foregoing buffering agents may include an alkali metal or an alkaline earth metal.

In another embodiment, the present disclosure provides a method for producing a medical product. The method comprises providing a solution comprising a solid-state argatroban dissolved in an aqueous solution and applying the solution to a surface of a medical device. The medical product is selected from the group consisting of a transdermal patch, a stent, a polymeric substrate and combinations thereof. Application of the solution on the medical device may be by way of spraying the solution onto one or more surfaces of the medical device. Alternatively, the medical device may be immersed into the solution. In an alternative embodiment, the method includes drying the applied solution and forming a coating of argatroban on the medical device.

### EXAMPLES

By way of example and not limitation, the following examples are illustrative of various embodiments of the present disclosure.

### Example 1

Preparation of concentrated solid-state salt formulations of argatroban without bulking agents

| | |
|---|---|
| Argatroban | 10-550 mg |
| Buffers inorganic acids (Glacial Acetic Acid) | 0.172 mL (3 N) |
| NaOH | To adjust pH as required |
| Water for Injection, USP | q.s. to 1 mL |
| Lyophilized (annealing) | Solid-state (powder) |
| Argatroban | 50 mg |
| Buffers inorganic acids Hydrochloric Acid, | 0.0083 mL |
| NF (37%) | |
| NaOH | To adjust pH as required |
| Water for Injection, USP | q.s. to 1 mL |
| Lyophilized (annealing) | Solid-state (powder) |
| Argatroban | 10-550 mg |
| Buffers inorganic acids (Glacial Acetic Acid) | 0.172 mL (3 N) |
| NaOH | To adjust pH as required |
| Water for Injection, USP | q.s. to 1 mL |
| Precipitation | Solid State (powder) |
| Argatroban | 10-550 mg |
| Buffers inorganic acids (Glacial Acetic Acid) | 0.172 mL (3 N) |
| Methanol and methylene chloride | 10:20:80 (v/v) |
| NaOH | To adjust pH as required |
| Water for Injection, USP | q.s. to 1 mL |
| Precipitation | Solid State (powder) |
| Argatroban | 35 mg |
| Buffers inorganic acids citric Acid, NF (37%) | 0.053 mL |
| NaOH | To adjust pH as required |
| Water for Injection, USP | q.s. to 1 mL |
| Lyophilized (annealing) | Solid-state (powder) |

Suitable equipment and glassware for compounding, filtering and filling were properly washed and depyrogenated. The filter assembly, filling tube assembly and other parts and equipment were sterilized. Eighty percent (80%) of the final volume of cool Water for Injection was brought to room temperature in the compounding tank. An appropriate acid was added to the tank and the solution was stirred until completely dissolved. Argatroban was added in the final step, and tank was adjusted to 100% of the final volume with Water for Injection. The solution was stirred until all the components were completely dissolved, and the pH adjusted between 4.0-5.0. The solution was filtered and aseptically filled into glass vials. The filled vials were partially closed within the lyophilization stoppers and then placed into the lyophilization chambers. The following general lyophilization cycle parameters were used as described in Table 1 and were based on the prior determined Tg of the solutions:

TABLE 1

| Shelf Temp | | Time (min.) | Ramp Rate | Vacuum (mT) |
|---|---|---|---|---|
| 2.0-5.0° | C. | — | — | — |
| −40.0° | C. | 480 | 1 degree/min | Initial freeze |
| −35.0° | C. | 360 | 0.5 degree/min | 100 |
| −25.0° | C. | 6000 | 1 degree/min | 100 |
| 20.0° | C. | 1260 | 1 degree/min | 100 |

After completion of the freeze drying cycle, the stoppers were fully closed prior to removal of the vials from the lyophilization chamber. The dry vials were then removed from the chamber and sealed with aluminum seals.

The samples (dry cake) were subjected to a battery of tests (X-ray powder diffraction, Thermogravimetric analysis (TG), Karl-Fischer titration and Differential scanning calorimetry (DSC)). At pre-determined intervals, samples were reconstituted in 5-mL water and then tested.

### Example 2

Preparation of concentrated solid-state salt formulations of argatroban with bulking agents

| | |
|---|---|
| Argatroban | 10-550 mg |
| Buffers inorganic acids (Glacial Acetic Acid) | 0.172 mL (3 N) |
| Sodium Acetate Trihydrate, USP | 2.8 mg |
| Lactose, USP | 150 mg |
| Acetic acid or NaOH | To adjust pH as required |
| Water for Injection, USP | q.s to 1 mL. |
| Lyophilized (annealing) | Solid-state (powder) |
| Argatroban | 50 mg |
| Buffers inorganic acids Hydrochloric Acid, | 0.0083 mL |
| NF (37%) | |
| Sodium Acetate Trihydrate, USP | 2.8 mg |
| Manitol, USP | 50 mg |
| Acetic acid or NaOH | To adjust pH as required |
| Water for Injection, USP | q.s to 1 mL. |
| Lyophilized (annealing) | Solid-state (powder) |
| Argatroban | 35 mg |
| Buffers inorganic acids citric Acid, NF (37%) | 0.053 mL |
| Sodium Acetate Trihydrate, USP | 2.8 mg |
| Sucrose, USP | 25 mg |
| Acetic acid or NaOH | To adjust pH as required |
| Water for Injection, USP | q.s to 1 mL. |
| Lyophilized | Solid-state (powder) |

The suitable equipment and glassware for compounding, filtering, and filling was properly washed and depyrogenated. The filter assembly, filling tube assembly and other parts and equipment were sterilized. Eighty percent (80%) of the final volume of cool water for Injection was brought to room temperature in the compounding tank. An appropriate acid was added to the tank, and the solution was stirred until completely dissolved. Argatroban was added and stirred until completely dissolved. A bulking agent was added in the final step, and tank was adjusted to 100% of the final volume with Water for Injection. The solution was stirred until the bulking agent was completely dissolved, and the pH adjusted between 4.0-5.0. The solution was filtered and aseptically filled into glass vials. The filled vials were partially closed with the lyophilization stoppers and then placed into the lyophilization chambers. The general lyophilization cycles parameters were used as described in Table 2 and were based on the prior determined Tg of the solutions:

TABLE 2
For solutions annealed

| Shelf Temp | | Time (min.) | Ramp Rate | Vacuum (mT) |
|---|---|---|---|---|
| 2.0-5.0° | C. | — | — | — |
| −40.0° | C. | 480 | 1 degree/min | Initial freeze |
| −35.0° | C. | 360 | 0.5 degree/min | 100 |
| −25.0° | C. | 6000 | 1 degree/min | 100 |
| 20.0° | C. | 1260 | 1 degree/min | 100 |

TABLE 3
For solutions not annealed

| Shelf Temp | | Time (min.) | Ramp Rate | Vacuum (mT) |
|---|---|---|---|---|
| 2.0-5.0° | C. | — | — | — |
| −40.0° | C. | 480 | 1 degree/min | Initial freeze |
| −25.0° | C. | 6000 | 1 degree/min | 100 |
| 20.0° | C. | 1260 | 1 degree/min | 100 |

After completion of the freeze drying cycle, the stoppers were fully closed prior to removal of the vials from the lyophilization chamber. The dry vials were then removed from the chamber and sealed with aluminum seals.

The samples (dry cake) were subjected to a battery of tests (X-ray powder diffraction Thermogravimetric analysis (TG), Karl-Fischer titration and Differential scanning calorimetry (DSC)). At pre-determined intervals, samples were reconstituted in 5-mL water and then tested.

### Results

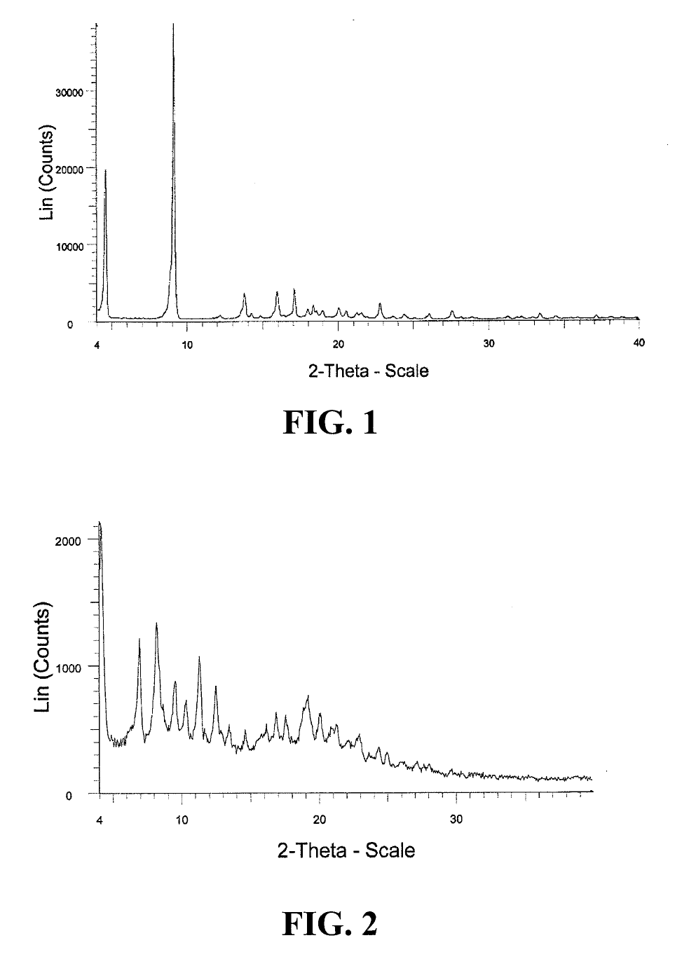

A crystalline form of (±)-1-[5-[(aminoiminomethyl)amino]-1-oxo-2-[[(1,2,3,4-tetrahydro-3-methyl-8-quinolinyl) sulfonyl]amino]pentyl]-4-methyl-2-piperidinecarboxylic acid monohydrate (using water and ethanol to crystallize the free base) exhibits a characteristic X-ray powder diffraction pattern with characteristic peaks expressed in d-values (Å): 19.42 (vs), 9.74 (vs), 7.29 (vw), 6.43 (m), 6.22 (vw), 5.98 (vw), 5.56 (m), 5.18 (m), 4.94 (w), 4.83 (w), 4.69 (vw), 4.43 (vw), 4.32 (w), 4.18 (vw), 4.12 (vw), 3.90 (vw), 3.75 (vw), 3.64 (vw), 3.41 (w), 3.23 (w), 3.16 (vw), 3.08 (vw), 2.85 (vw), 2.80 (vw), 2.77 (vw), 2.67 (vw),2.60 (vw), 2.53 (vw), 2.49 (vw), 2.41(vw), 2.35 (vw), designated as Form A. The abbreviations in brackets mean: (vs)=very strong intensity; (s)=strong intensity; (m)=medium intensity; (w)=weak intensity; and (vw)=very weak intensity. A corresponding X-ray powder diffraction pattern for this form is depicted in FIG. 1

A crystalline form of (±)-1-[5-[(aminoiminomethyl)amino]-1-oxo-2-[[(1,2,3,4-tetrahydro-3-methyl-8-quinolinyl) sulfonyl]amino]pentyl]-4-methyl-2-piperidinecarboxylic acid monohydrate (using water and methylene chloride, methanol, ethanol to crystallize the free base) exhibits a characteristic X-ray powder diffraction pattern with characteristic peaks expressed in d-values (Å): 20.88 (w), 19.31 (vs), 10.45 (w), 9.66 (vs), 7.64 (vw), 7.26 (vw), 6.42 (m), 6.22 (vw), 5.96 (vw), 5.55 (m), 5.17 (w), 4.64 (vw), 4.83 (w), 4.69 (vw), 4.43 (vw), 4.18 (vw), 4.12 (vw), 3.90 (vw), 3.75 (vw), 3.64 (vw), 3.43 (vw), 3.22 (vw), 2.80 (vw), 2.77 (vw), 2.67 (m),2.59 (vw), 2.53 (vw), 2.46 (vw), 2.41(vw), 2.35 (vw),designated as Form B. The abbreviations in brackets mean: (vs)=very strong intensity; (s)=strong intensity; (m)=medium intensity; (w)=weak intensity; and (vw)=very weak intensity. A corresponding X-ray powder diffraction pattern for this form is depicted in FIG.

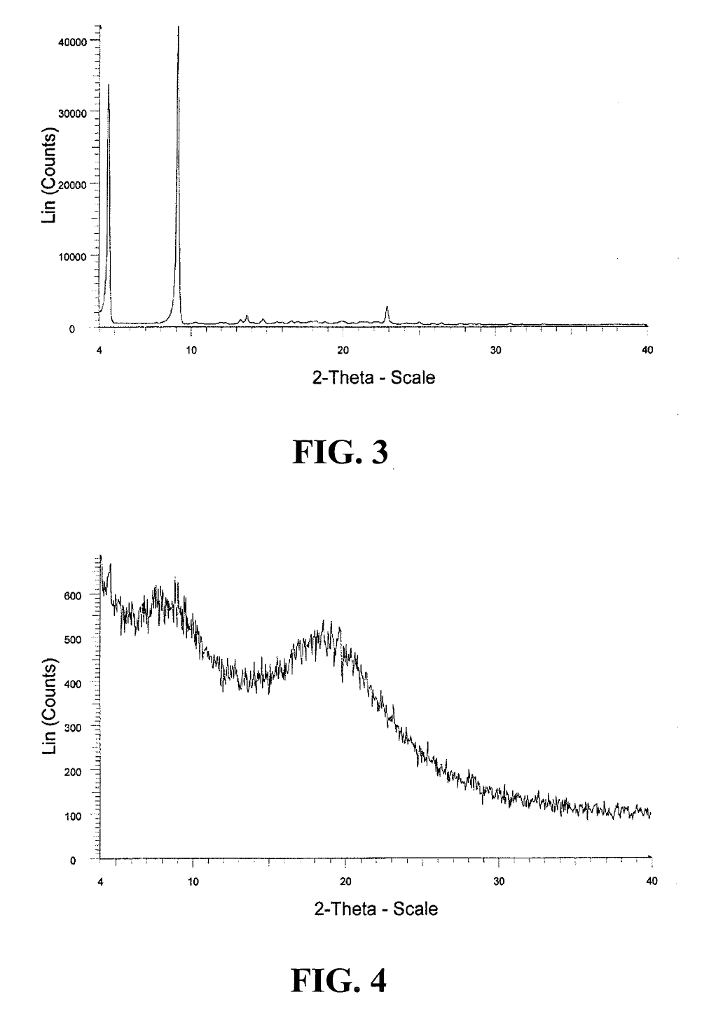

A crystalline hydrochloride salt form of (±)-1-[5-[(aminoiminomethyl)amino]-1-oxo-2-[[(1,2,3,4-tetrahydro-3-methyl-8-quinolinyl) sulfonyl]amino]pentyl]-4-methyl-2-piperidinecarboxylic acid hydrate (crystallized by lyophilization) exhibits a characteristic X-ray powder diffraction pattern with characteristic peaks expressed in d-values (Å): 19.46 (vs), 9.74 (vs), 8.63 (vw), 7.42 (vw), 6.99 (vw), 6.50 (w), 6.02 (w), 5.53 (vw), 5.35 (vw), 5.20 (vw),4.86 (vw), 4.71 (w), 4.45 (vw), 4.17 (vw), 3.99 (vw), 3.88 (m), 3.56 (vw), 3.44 (w), 3.36 (w), 3.22 (vw), 2.88 (vw), designated as Form C. The abbreviations in brackets mean: (vs)=very strong intensity; (s)=strong intensity; (m)=medium intensity; (w)=weak intensity; and (vw)=very weak intensity. A corresponding X-ray powder diffraction pattern for the crystalline hydrochloride salt form of argatroban is depicted in FIG. 3.

An amorphous acetate salt form of (±)-1-[5-[(aminoiminomethyl)amino]-1-oxo-2-[[(1,2,3,4-tetrahydro-3-methyl-8-quinolinyl) sulfonyl]amino]pentyl]-4-methyl-2-piperidinecarboxylic acid hydrate (amorphous by lyophilization) exhibits no characteristic X-ray powder diffraction pattern. A corresponding X-ray powder diffraction pattern for the amorphous acetate salt form of argatroban is depicted in FIG. 4

The solid-state salt formulations of argatroban described in Examples 1-2 can be added to a solution (e.g. aqueous) in any suitable amount to make a final argatroban solution that can be administered. The solutions can be sterilized prior to use. The final solutions can contain the solid-state salt formulations of argatroban in an amount ranging from 1-1000 mg/mL, preferably 1-100 mg/mL. Pharmaceutically acceptable acids and solvents can be utilized to control the solubility and stability of the formulation. Suitable osmotic-adjusting agents may be added as known in the art ranging from 1-250 mg/mL. The solutions of the present disclosure can be packaged in sealed containers and made sterile by aseptic processes.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A composition comprising a solid-state argatroban that redissolves in a solution.

2. The composition of claim 1, wherein the solution is selected from the group consisting of an aqueous solution, an injection diluent and combinations thereof.

3. The composition of claim 1, wherein the solid-state argatroban comprises a powdered form.

4. The composition of claim 1, wherein the solid-state argatroban comprises a crystalline form.

5. The composition of claim 1, wherein the solid-state argatroban comprises a stable crystalline form and maintains the isomeric composition of argatroban.

6. The composition of claim 1, wherein the solid-state argatroban comprises a metastable crystalline form and maintains the isomeric composition of argatroban.

7. The composition of claim 1, wherein the solid-state argatroban comprises an amorphous form.

8. The composition of claim 1, wherein the solid-state argatroban comprises a stable amorphous form and maintains the isomeric composition of argatroban.

9. The composition of claim 1, wherein the solid-state argatroban comprises a mixture of an amorphous and crystalline powdered form.

10. The composition of claim 1, wherein the solid-state argatroban comprises a mixture of an amorphous and crystalline powdered form and maintains the isomeric composition of argatroban.

11. The composition of claim 1, wherein the solid state argatroban is made by lyophilizing an argatroban salt solution.

12. The composition of claim 11, wherein the argatroban salt solution does includes at least one diluted acid.

13. The composition of claim 11, wherein the argatroban salt solution does includes at least one solvent.

14. The composition of claim 11, wherein the argatroban salt solution comprises a pH ranging from about 3.5 to about 8.5.

15. The composition of claim 11, wherein the argatroban salt solution comprises a pH ranging from about 4.5 to about 6.5.

16. The composition of claim 11, wherein the argatroban salt solution comprises a component selected from the group consisting of sodium chloride, calcium chloride, potassium chloride, dextrose, sodium lactate and combinations thereof.

17. The composition of claim 11, wherein the argatroban salt solution comprises a component selected from the group consisting of lactose, manitol maltose, sucrose, dextrans, glycine, sorbitol, glucose, calcium, non-reducing sugars and combinations thereof.

18. The composition of claim 11, wherein the argatroban salt solution has a shelf life greater than about three weeks.

19. The composition of claim 11, wherein the argatroban salt solution is sterilized.

20. A reconstitutable compound comprising a solid-state argatroban having an X-ray diffraction pattern having a characteristic peak expressed in d-values (Å) selected from the group consisting of: 19.46, 9.74, 8.63, 7.42, 6.99, 6.50, 6.02, 5.53, 5.35, 5.20, 4.86, 4.71, 4.45, 4.17, 3.99, 3.88, 3.56, 3.44, 3.36, 3.22, 2.88.

21. An argatroban composition comprising an X-ray diffraction pattern having a characteristic peak expressed in d-values (Å) selected from the group consisting of: 19.46, 9.74, 8.63, 7.42, 6.99, 6.50, 6.02, 5.53, 5.35, 5.20, 4.86, 4.71, 4.45, 4.17, 3.99, 3.88, 3.56, 3.44, 3.36, 3.22, 2.88.

22. A method for producing a reconstituted argatroban solution, the method comprising:
dissolving a solid-state argatroban in a solution, the solution having an argatroban concentration ranging between about 0.5 μg/mL to about 500 mg/mL.

23. A method for producing a medical product, the method comprising:
providing a solution comprising a solid-state argatroban dissolved in an aqueous solution; and
applying the solution to a surface of a medical device.

24. The method of claim 23, wherein the aqueous solution comprises an argatroban concentration ranging between about 0.5 μg/mL to about 500 mg/mL

25. The method of claim 23, wherein the medical product is selected from the group consisting of a transdermal patch, a stent, a polymeric substrate and combinations thereof.

26. The method of claim 23 comprising drying the solution and forming a coating of argatroban on the medical device.

27. A method of administering argatroban to a person, the method comprising:
preparing a solution comprising a solid-state argatroban dissolved in an aqueous solution, the solution having an argatroban concentration ranging between about 0.5 μg/mL to about 500 mg/mL;
diluting the solution; and
administering the solution to the person.

28. The method of claim 27, wherein the solution is sterilized before administering to the person.

29. A method of providing anticoagulation or antithrombotic therapy to a person, the method comprising:
administering to a person in need of same a solution comprising a solid-state argatroban dissolved in an aqueous solution, the solution having an argatroban concentration between about 0.5 μg/mL to about 500 mg/mL.

30. A method of making a solid-state argatroban, the method comprising:
dissolving an acid in an aqueous solution;
dissolving argatroban in free base form in the acidified aqueous solution;
filtering the dissolved argatroban solution; and
freeze drying the filtered argatroban solution to form the solid-state argatroban.

31. The method of claim 30 comprising adjusting the pH of the dissolved argatroban solution to between 4.0 and 5.0 before filtering the dissolved argatroban solution.

32. The method of claim 30 comprising adding a bulking agent to the dissolved argatroban solution before filtering the dissolved argatroban solution.

33. The method of claim 32, wherein the bulking agent is selected from the group consisting of lactose, sucrose, manitol, sugars, non-reducing sugars and combinations thereof.

34. The method of claim 30 comprising dissolving the solid-state argatroban in an aqueous solution to form a reconstituted argatroban solution.
